(19) United States
(12) Patent Application Publication
(10) Pub. No.: US 2005/0171004 A1
(43) Pub. Date: Aug. 4, 2005

(54) Use of cardiotrophin-1 to promote wound healing and counteract overt fibrosis

(76) Inventors: Darren H. Freed, Manitoba (CA); Ian M.C. Dixon, Manitoba (CA); W. R. Giles, Manitoba (CA)

Correspondence Address: ADE & COMPANY, 1700-360 MAIN STREET, WINNIPEG, MB, R3C3Z3, CA

(21) Appl. No.: 10/954,159

(22) Filed: Sep. 30, 2004

(30) Foreign Application Priority Data
Sep. 30, 2003 (CA) 2,443,423

(51) Int. Cl. A61K 38/17

(52) U.S. Cl. 514/2

(57) ABSTRACT

The use of CT-1, bioactive fragments thereof and mimetics thereof in promoting wound healing is described herein.

### FIELD OF THE INVENTION

The present invention relates generally to the field of pharmaceutical compounds and medical treatments. More specifically, the present invention relates to the use of cardiotrpohin-1 (CT-1) to promote wound healing.

### BACKGROUND OF THE INVENTION

Patients suffering coronary artery disease are at risk of suffering a heart attack or myocardial infarction (MI). Despite recent advances in treatments for cardiac patients, most who survive the acute complications of a large MI go on to develop congestive heart failure from one to five years thereafter. Congestive heart failure is named for hemodynamic complications arising from lack of forward output of the left ventricle, and is due to pooling of blood and subsequent seepage of plasma from the microcirculation to the extracellular space i.e., lungs, ankles, etc. The classic definition of heart failure is a lack of forward output from the heart that is commensurate with the metabolic demands of the body. The prognosis for these patients is grave indeed, and remains so. Thus a clear challenge to the researcher in basic cardiovascular sciences is to pursue avenues or mechanisms to alleviate the morbidity and mortality that follows a large heart attack.

During the development of congestive heart failure, altered expression of several hormones and related cytokines occurs that confer remodeling of the surviving or remnant heart tissue and excessive healing of the cardiac infarct scar, including excessive growth of remnant myocytes (cardiac hypertrophy) with attendant collagen deposition and fibrosis of the spaces between myocytes. This over-secretion and over-deposition of collagen (types I and III in particular) is linked to eventual inappropriate physical stiffening of the entire post-MI heart.

The events that lead to cardiac fibrosis are mediated by specialized non-muscle cells i.e., cardiac fibroblasts (largely quiescent) and cardiac myofibroblasts (hypersecretory and muscular phenotypic variants of fibroblasts). Cardiac myofibroblasts are unique cells; some have described them as half-way between vascular smooth muscle cells and fibroblasts. It is these cells that are responsible for the excessive collagen accumulation and fibrosis that follows MI. By tethering themselves to the surrounding matrix, they may exert tension on the matrix, thereby inducing scar thinning which impairs overall ventricular function. Myofibroblasts are also responsible for fibrosis of other organs, such as skin (ie scleroderma), lung (ie pulmonary fibrosis) and kidney (ie renal fibrosis and kidney failure). As such they are critical mediators for not only normal wound healing, but also pathologic fibrosis of non-injured organs. Clearly the modulation of this cell type is a potential therapeutic target in treating the organ fibrosis that follows normal wound healing and also the pathologic activation of these cells in fibro-proliferative diseases.

### SUMMARY OF THE INVENTION

According to a first aspect of the invention, there is provided a method of promoting wound healing comprising administering to a patient in need thereof an effective amount of CT-1, a bioactive fragment of CT-1 or a mimetic thereof.

According to a second aspect of the invention, there is provided a pharmaceutical composition comprising an effective amount of CT-1, a bioactive fragment of CT-1 or a mimetic thereof.

According to a third aspect of the invention, there is provided the use of an effective amount of CT-1, a bioactive fragment of CT-1 or a mimetic thereof for promoting wound healing.

### BRIEF DESCRIPTION OF THE DRAWINGS

FIG. 1: Expression of CT-1 at 24 & 48 hr, 2, 4 and 8 weeks after MI. Cardiotrophin-1 expression was determined in 50 μg cardiac tissue lysate by Western analysis with anti-CT-1 monoclonal antibody. Samples from 3 separate animals were quantified and relative intensity is expressed in arbitrary densitometric units (mean±SEM).

FIG. 2. Graphic representation of CT-1 expression after permanent coronary artery ligation in the rat in the infarct zone and in viable/remnant myocardium.

FIG. 3: Expression of α smooth muscle actin after isolation of primary adult rat cardiac fibroblasts.

FIG. 4. CT-1 Inducted phosphorylation of gp130 and Jak1/2. Panel A. Phosphorylation of gp130 was investigated by immunoprecipitating gp130 from cell lysates of CT-1 treated or non-treated cells and probing with anti-phosphotyrosine antibody. To verify equal protein loading between different lanes, the membrane was stripped and probed for gp130. Panel B. Jak1 and Jak2 phosphorylation was analyzed by western analysis. Equal protein loading was verified by stripping the membranes and probing for total Jak1 and total Jak2. Representative western blots from 3 separate experiments are shown.

FIG. 5. CT-1 Induced Phosphorylation of Signaling Pathways. Panel A. CT-1 induces phosphorylation of STAT1 and STAT3. To verify equal protein loading, the membranes were stripped and probed for total STAT3. Panel B. CT-1 induces phosphorylation of ERK1/2, p38 MAPK, JNK and Src. To verify equal protein loading, the membranes were stripped and probed for actin. Representative western blots from 3 separate experiments are shown.

FIG. 6. CT-1 induces nuclear accumulation of STAT3. Cells were stimulated with CT-1 for 5 minutes and immunostained with anti-STAT3 antibody. Nuclei were identified by staining with Hoechst 33342 (lower panels). Representative images are shown from 3 separate experiments.

FIG. 7: CT-1 induces cardiac fibroblast DNA synthesis. DNA synthesis was measured by incorporation of 3H-thymidine. Cardiac fibroblasts were incubated in serum-free media from time zero, then pulse labeled with 3H-thymidine for 30 minutes. At 24 hours, cells were stimulated with 10 ng/mL CT-1, or further incubated in serum-free media. Samples from 7 separate experiments were analyzed in triplicate. Results are displayed as mean±SEM. *p<0.05 vs serum-free at the same time point.

FIG. 8: Maximal DNA synthesis occurs with 10-20 ng/mL CT-1. Cardiac fibroblasts were incubated with CT-1 at specified concentrations for 6 hours (A) or 12 hours (B) and labeled with 3H-thymidine for 30 minutes. Samples from 7 separate experiments were analyzed in triplicate. Results are displayed as mean±SEM. *p<0.05 vs non-stimulated control.

FIG. 9: CT-1 induced DNA synthesis is dependent on the Jak/STAT, MAPK, P13K and Src pathways. Cardiac fibroblasts were pretreated with inhibitors at specified concentrations, incubated with CT-1 for 12 hours and labeled with 3H-thymidine for 30 minutes. Samples from 7 separate experiments were analyzed in triplicate. *p<0.05 vs CT-1 10 ng/mL.

FIG. 10: CT-1 induces expression of cyclins and Rb phosphorylation. Cardiac fibroblasts were incubated with CT-1 for specified times. Cell lysates were subjected to Western analysis with antibodies against cyclins A, E, Rb pS795, and pS807/811. Representative blots from 3 separate experiments is shown.

FIG. 11. CT-1 induces nuclear accumulation of PCNA. Cardiac fibroblasts were incubated with CT-1 at specified concentrations for 24 hours, fixed and immunostained with anti-PCNA antibody. The number of nuclei positive for PCNA are expressed as a ratio with total nuclei. *p<0.05 vs non-stimulated control.

FIG. 12: CT-1 increases total cell number. Equal numbers of cardiac fibroblasts were loaded into 24 well plates and stimulated with CT-1 for 24 hours at specified concentrations. The cells were trypsinized, diluted in filtered phosphate buffered saline and counted with a Coulter counter. *p<0.05 vs non-stimulated control.

FIG. 13. CT-1 increases protein synthesis. Protein synthesis was measured by incorporation of 3H-leucine. Results are displayed as mean±SEM. Samples from 5 separate experiments were analyzed in triplicate. *p<0.05 vs control.

FIG. 14. CT-1 increases phosphorylation of protein synthesis regulatory proteins. Membranes were probed with phospho-specific antibodies. To verify equal protein loading, the membranes were stripped and probed for actin. Representative blots from 3 separate experiments are shown.

FIG. 15. Incubation with inhibitors of signaling pathways depresses protein synthesis. Protein synthesis was measured by incorporation of 3H-leucine. Results are displayed as mean±SEM. Samples from 3 separate experiments were analyzed in triplicate. *p<0.05 vs control. #p<0.05 vs CT-1 10 ng/mL.

FIG. 16. Incubation with inhibitors of signaling pathways depresses phosphorylation of translational regulatory proteins. Membranes were probed with phospho-specific antibodies. To verify equal protein loading, the membranes were stripped and probed for actin.

FIG. 17. Proposed scheme of the CT-1 signaling cascade in cardiac fibroblasts leading to induction of protein synthesis.

FIG. 18: Effect of CT-1 on mature collagen synthesis. Collagen synthesis was determined by radioimmunoassay for procollagen-1 carboxy-propeptide (P1CP) in conditioned media from cardiac fibroblasts stimulated with specified concentrations of CT-1 for 24 hours. Conditioned media from 3 separate experiments was analyzed in triplicate. Results are displayed as mean±SEM. Panel A shows the P1CP content of media collected from CT-1 stimulated cells. Panel B shows the P1CP concentration corrected for the number of cells at the end of the experiment. *p<0.05 vs non-stimulated control.

FIG. 19: CT-1 is a chemoattractant for rat cardiac fibroblasts. The ability of CT-1 to induce cell migration was analyzed with a Boyden chamber. Results displayed represent the number of cells that migrated through the membrane separating the cells and media containing CT-1. Cardiac fibroblasts were incubated with CT-1 at specified concentrations overnight. *p<0.05 vs non-stimulated control.

FIG. 20: CT-1 induced chemotaxis is dependent on signaling pathways. Signaling pathways utilized in CT-1 induced chemotaxis were examined using specific inhibitors. Cells that had migrated through the membrane were stained and counted. *p<0.05 vs non-stimulated control. # p<0.05 vs CT-1.

FIG. 21: CT-1 induced chemotaxis is dependent on integrins. The role of extracellular matrix interactions utilized in CT-1 induced chemotaxis were examined using integrin inhibitors. Cells that had migrated through the membrane were stained and counted. *p<0.05 vs non-stimulated control. # p<0.05 vs CT-1.

FIG. 22: Cardiotrophin-1 induces accumulation of cortactin at the leading edge. Localization of the actin stabilizing protein, cortactin, was examined using the “wounded monolayer” model. Cells were immunostained for cortactin 4 hours after the creation of the wound. Green: cortactin. Red: Actin (rhodamine phalloidin). Blue: Nuclei.

FIG. 23: CT-1 induced migration is dependent on ion channel and myosin light chain kinase function. Cells that had migrated through the membrane were stained and counted. *p<0.05 vs non-stimulated control. # p<0.05 vs CT-1.

FIG. 24. Cardiotrohpin-1 induces hyperpolarization of cardiac fibroblasts. Membrane potential was estimated with the use of DiBac4(3) voltage sensitive dye. Results displayed are representative of 3 separate experiments.

FIG. 25: CT-1 induces phosphorylation of myosin light chain. Panel A: Cardiac fibroblasts were stimulated with CT-1 (10 ng/mL) for specified times. Panel B: Cardiac fibroblasts were treated for 30 minutes and lysates were analyzed for myosin light chain phosphorylation. The blots were stripped and reprobed for actin to verify equal protein loading. Representative blots from three separate experiments are shown.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “wound” refers to an injury to a body causing laceration and/or damage to underlying tissues.

As used herein, “bioactive fragment” refers to a fragment, mutant or variant of a peptide which retains the biological activity of interest of the native, full-length peptide.

As used herein, “effective amount” refers to an amount that is sufficient to achieve the desired result. In regard cardiotrophin-1 (CT-1), “effective amount” refers to an amount which is sufficient to promote wound healing, that is, to attract bone marrow stem cells and/or myofibroblasts to a wound site.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, the term “treating” in its various grammatical forms refers to preventing, curing, reversing, attenuating, alleviating, minimizing, suppressing or halting the deleterious effects of a disease state, disease progression, disease causitive agent other abnormal condition.

Described herein is the use of CT-1, bioactive fragments thereof and mimetics thereof in promoting wound healing.

Circulating pluripotent progenitor cells of bone marrow origin are known to migrate to the heart after myocardial infarction. Bone marrow stem cells (BMSCs) are pleuripotent and it is likely that these cells may differentiate a) to fibroblasts and/or myofibroblasts to effect wound healing, or b) myocytes to effect cardiac regeneration. Our studies indicate that CT-1 serves as the cytokine “beacon” guiding the homing of pleuripotent stem cells to the site of injury. A recent study (P. Anversa and colleagues, New York Medical College) indicates that these cells rapidly migrate to the infarcted myocardium and participate in cardiac regeneration i.e., new myocytes formed, while Ray Chiu (McGill and Taipei, Thailand) and coworkers have shown that these cells migrate to the site of infarction i.e., the infarct scar, and that they take on a myofibroblastic, or in some cases, a cardiomyocyte phenotype. These cells have also been observed in the actual site of the incision made while surgically infarcting the animal (data not published), demonstrating that they are involved in non-cardiac wound healing as well. To date the specific stimulus for migration of these cells to the site if injury is not known. In either case, we believe that a critical factor mediating this movement of BMSCs is the localized overexpression of CT-1 by cells in the infarct scar.

CT-1 is a cytokine which is unusual for its rapid rise, but more particularly, for its maintained expression in the infarct scar of post-MI heart.

CT-1 was cloned by Pennica et al in 1995 (Pennica D et al., Proc Natl Acad Sci 92, 1142-1146). It has 201 amino acids as a mature protein, is not glycosylated, has two cysteine residues, and carries 3 exons in an mRNA that is about 1.7 kb. The CT-1 gene is located on X′m 16 p11.1-11.2. In the CT-1 receptor, the residues crucial for ligand binding are located in the cytokine-binding module (CBM) which consist of two fibronectin-type-III like domains “of which the N-terminal domain contains a set of four conserved cysteine residues and the C-terminal domain a WSXWS motif”. The CT-1 ligand may have a similarly conserved binding region. Mutation analysis studies of IL-6 seems to verify this assumption. Amino acid sequence similarities were provided as evidence that this gp130 signaling cytokine is a member of the LIF or IL-6 superfamily, and that it is 21.5 kDa in size. One general complication in dealing with pleiotrophic cytokines is redundancy of cytokine actions. This may not apply however to CT-1. Important differences in receptor usage distinguish the transduction of signaling by CT-1, compared to other members of the IL-6 superfamily. For example, the unique combination of its receptor (an LIF/gp130 dimer) means that only LIF and CT-1 share a receptor, and that LIF does not share the bioactive properties of CT-1.

Other cytokines commonly associated with wound healing i.e., TGF-β are profibrotic, abrogate proliferation of myofibroblasts, and may upregulate of cellular tethering and scar contraction via enhanced focal adhesion function. Finally, TGF-β may stimulate the phenotypic conversion of fibroblast to myofibroblast. It is now clear that CT-1 exerts opposite effects. CT-1's role in post-MI hearts is much less well known than that of TGF-β, but our recent published data indicates that it is upregulated 24 h after MI and is maintained beyond the point of decompensated heart failure (8 weeks) in our experimental animal model. Thus, CT-1 actions are fundamentally different from those of TGF-β, particularly in regulation of proliferation, migration, and effect on normalized collagen synthesis by cardiac myofibroblasts.

Described herein is the use of CT-1 and its mimetics in wound healing that involves myofibroblast and/or BMSC participation. CT-1 is a strong chemoattractant for both cell types, is a proliferative agent, is an anti-fibrotic, and may complement or even counteract TGF-β's effects, depending on whether the specific mode of wound healing is in physiological or pathophysiological boundaries per se. Finally, we have insight into how CT-1 may mediate its effects by noting its hyperpolarizing effect and influence on Ca-dependent proteins that regulate of myosin light chain kinases in myofibroblasts. Presumably changes in the latter effect myofibroblast migration by modulating myosin function and thus cellular contractility.

Also described herein is the use of cardiotrophin-1 (CT-1), and other synthetic compounds that activate the gp130 signaling cascade to modulate key aspects of wound healing, not only in heart but wound healing in other tissues.

Fibrosis stems from wound healing gone awry, thus the term “hypertrophic” scars has recently come to the fore. As will be apparent to one of skill in the art, wound healing applies to skin, lung, liver, heart, teeth and gums, and perhaps skeletal muscle. Myofibroblasts are at the core of this process; they disappear in normal wound healing by apoptotic mechanisms. They persist for decades however in post-MI heart and its likely that this is the basis of so-called hypertrophic scarring.

It is emphasized that although we are focused on cardiac wound healing per se, our work would indicate a role for CT-1 in a wide spectrum of wound healing. However, CT-1 (or mimetics) can be used to induce proliferation and migration of fibroblasts, and antagonize the conversion to a myofibroblast phenotype and over-expression of mature collagen. Secondly, CT-1 (or mimetics) can be used to induce migration of bone marrow stem cells.

The invention will now be described by way of examples; however, the invention is not limited by the examples which are provided for illustrative purposes.

Materials and Methods

Myocardial Infarction in Rats

All experimental protocols for animal studies were approved by the Animal Care Committee of the University of Manitoba, following guidelines set forth by the Canadian Council on Animal Care. MI was produced in 200-250 gm anesthetized (0.01-0.05 mg/kg buprenorphine subcutaneous premedication, 2-2.5% isofluorane inhalation anesthetic) male Sprague-Dawley rats by ligation of the left coronary artery as previously described (Dixon et al., 1990, Circ Res 66: 782-788). The mortality rate associated with this procedure was ˜40% within 48 hours and the resultant scar occupied ˜40% of the left ventricle. Sham operated animals received the same procedure, lacking only the coronary artery ligature, and were used for comparative purposes. Animals were sacrificed at specified time points and tissue was isolated from two regions of the left ventricle: remnant/viable and infarct zone/scar. These specimens were pulverized under liquid nitrogen, homogenized, and lysed in 2× sodium dodecyl sulfate (SDS) buffer (0.125 M Tris, 2% SDS, 20% glycerol) at 4° C. for 60 minutes then sonicated for 15 seconds. Insoluble material was removed by centrifugation and the clear supernatant was used for Western analysis of CT-1 expression.

Isolation and Characterization of Cardiac (Myo)Fibroblasts

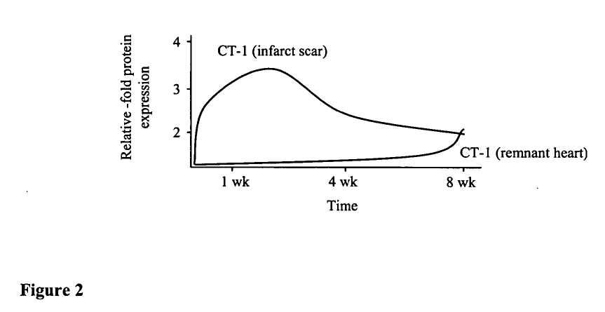

Fibroblasts were isolated from the hearts of 200-250 g adult male Sprague-Dawley rats as previously described (Hao et al., 2000, Am J Physiol Heart Circ Physiol 279: H3020-H3030). Briefly, hearts were subjected to Langendorff perfusion at 37° C. with Joklik's medium containing 0.1% collagenase (Worthington Biochemical Corporation, Lakewood, N.J.) for 20-25 minutes. Collagenase was neutralized by addition of an equal volume of DMEM/F12 medium containing 10% FBS and liberated cells were collected by centrifugation at 500×g for 5 minutes. Cells were resuspended in fresh DMEM/F12 containing 10% FBS and plated on 75 cm² culture flasks at 37° C. with 5% CO₂ for 3 hours. Non adherent cells (myocytes) were removed by changing the culture media and adherent cells (mainly fibroblasts) were incubated in DMEM/F12 containing 10% FBS, 100 U/mL penicillin, 100 μg/mL streptomycin, and 100 μM ascorbate. Fibroblasts were used for experiments after the second passage and the purity of these cells was ≧95%, using routine phenotyping methods as previously described (Peterson et al., 1999, Cardiovasc Res 41: 575-585). FIG. 2 demonstrates that these cells begin to express α-smooth muscle actin by the first passage, indicating that these cells are myofibroblasts. Cells were rendered quiescent by incubation in serum-free medium for 24 hours then stimulated with CT-1 for specified times and lysed in RIPA buffer (150 mM NaCl, 1% Triton X-100, 0.5% deoxycholate, 0.1% SDS, 50 mM Tris) with 1× protease inhibitor cocktail (Sigma-Aldrich, Oakville, Ontario) and 10 mM NaF, 1 mM Na₃VO₄ and 1 mM EGTA.

Western Analysis

Protein concentrations of tissue and cell lysates were determined by the BCA method (Smith et al., 1985, Anal Biochem 150: 76-85). Proteins were separated by 8-12% SDS-PAGE and transferred to PVDF membrane (Roche, Indianapolis, Ind.) for 1 hour at 300 mA for small proteins or for 2 hours at 500 mA for proteins greater than 100 kDa. Membranes were blocked with 5% non-fat skim milk in tris-buffered saline containing 0.2% Tween 20 (TBST). Proteins were visualized with ECL Plus (Amersham) after probing with primary and secondary antibodies. Band intensity was quantified using a CCD camera imaging densitometer (GS670, Bio-Rad Laboratories (Canada) Ltd. Mississauga, Ontario).

Immunofluorescence

Cardiac fibroblasts were seeded onto coverslips in 6 well dishes and allowed to attach overnight in media containing 10% serum. The cells were rendered quiescent in serum free media for 24 hours before being stimulated with CT-1 for specified times. The media was removed, cells were rinsed with PBS and fixed with 4% paraformaldehyde. Cells were permeabilized with 0.1% Triton X-100 and incubated with primary antibodies, biotinylated secondary antibodies, and streptavidin FITC. Nuclei were stained with Hoechst 33342. The cells were visualized with epifluorescent microscopy with appropriate filters (Nikon Canada).

Proliferation Assay

³H Thymidine Incorporation

DNA synthesis was measured by uptake of ³H-thymidine as previously described (Saward and Zahradka, 1997, Mol Cell Biochem 176: 53-59) with modification. Briefly, 2.5×10⁴ cells (counted with a hemacytometer) in DMEM/F12 with 10% FBS were loaded into each well of 24-well culture plates, allowed to attach overnight and rendered quiescent in serum-free DMEM with 100 μM ascorbate for 24 hours. S-phase re-entry in response to CT-1 was measured in two ways: pulse labeling with ³H-thymidine for 30 minutes every 6 hours, or labeling for the last 30 minutes of 6 or 12 hour incubation. DNA in cell lysate was precipitated with cold 20% trichloroacetic acid (TCA) and filtered through GF/A filters (Fisher). Beta emission from the dried filers was measured with Cytoscint scintillation fluid (ICN Pharmaceuticals, Costa Mesa, Calif.) and a scintillation counter (LS6500, Beckman Coulter, Fullerton, Calif.).

Cell Counting

To determine absolute cell numbers after stimulation with CT-1, the total number of cells were counted. Equal numbers of cells were loaded into each well of 24 well plates and incubated with CT-1 for 24 hours. The cells were detached with trypsin, diluted in filtered phosphate buffered saline, and counted with Model ZM Coulter cell counter (Beckman Coulter, Fullerton, Calif.).

Nuclear Accumulation of PCNA

Cardiac fibroblasts were seeded onto cover slips and allowed to attach overnight. The cells were incubated with serum free media for 24 hours prior to stimulation with CT-1 for an additional 24 hours. The cells were then fixed with cold acetone, permeabilized and immunostained for PCNA and counter stained with Hoechst 33342. Images from 5 high power fields per slide were captured and cells were counted. Nuclei that were positive for PCNA were counted and expressed as a ratio to total nuclei.

Protein Synthesis Assay

Protein synthesis was determined using the methods of Wolf and Neilson (Wolf and Neilson, 1990, Am J Physiol 259: 768-777). 2.5×10⁴ cells (counted with a hemacytometer) were loaded into each well of 24-well plates, allowed to attach overnight, then rendered quiescent in serum free DMEM/F12 for 24 hours. Cells were stimulated with CT-1 for an additional 24 hours in the presence of 2 μCi/mL ³H-leucine with or without inhibitors of signaling pathways. The culture media was aspirated, cells rinsed twice with phosphate buffered saline and proteins precipitated by two incubations with 10% TCA at room temperature. The precipitated protein was solubilized in 300 μl lysis buffer containing 0.5 M NaOH and 1% Triton X-100 at room temperature for 15 minutes. The lysate was transferred to scintillation vials, and beta emission was determined with 3 ml Ecolume scintillation fluid (ICN Pharmaceuticals) and a scintillation counter (LS6500, Beckman Coulter, Fullerton, Calif.).

Collagen Synthesis

Synthesis of mature type I collagen was determined by measuring the concentration of the carboxyterminal propeptide of type I collagen (P1CP) in conditioned media from CT-1 treated or untreated cardiac fibroblasts. 5×10⁴ cells were loaded into each well of a 6 well plate, allowed to attach overnight, and serum starved for 24 hours. Cells were treated with CT-1 at specified concentrations for an additional 24 or 48 hours. P1CP content in 0.1 mL conditioned media was determined by radioimmunoassay according to the manufacturer's specification (Diasorin Inc, Stillwater, Minn.). Radioactivity was quantified with a gamma counter (Gamma 5500, Beckman Coulter, Fullerton, Calif.) and the concentration of P1CP was determined by the % B/B₀ versus log concentration. This method has been shown to correlate well with ³H-proline incorporation into collagenase sensitive proteins (Nacher et al., 1999, Calcif Tissue Int 64: 224-228).

Migration Assay

Boyden Chamber

Migration of rat cardiac fibroblasts was measured using Boyden chamber (Neuro Probe Inc. Gaithersburg, Md.) assay (Richards and McCullough, 1984, Immunol Commun 13: 49-62). Chemoattractants were diluted in DMEM/F12 and loaded into the lower wells at specified concentrations. The polycarbonate membrane (5 μm pore) was placed over the wells, and 55 μL cell suspension in DMEM/F12 supplemented with 100 μM ascorbate was loaded into each well (1000 cells/mm²). The chamber was incubated overnight in 5% CO₂ and 100% humidity at 37° C. Cells that had migrated through the membrane and become adherent to the lower surface were fixed with methanol and stained with Diff-Quick stain (Dade Behring AG, Düdingen, Switzerland). Cell migration was determined by counting the number of cells per high power field.

Wounded Monolayer Model

Cardiac fibroblasts were seeded onto coverslips in 6-well dishes and allowed to proliferate. Once the cells were near confluency, they were serum deprived for 24 hours. A scrape was made down the middle of the coverslip with the blunt end of a 1 ml syringe and the media was changed again. Cells were further incubated in the presence or absence of CT-1 and inhibitors at specified concentrations for specified times. The cells were then subjected to immunofluorescence staining as described herein.

Estimation of Cardiac Myofibroblast Membrane Potential

Myofibroblast membrane potential was estimated using voltage sensitive DiBAC₄(3) dye (Molecular Probes, Eugene, Oreg.). DiBAC₄(3) is a potentiometric bisoxonol dye that partitions into the cell membrane and exhibits increased fluorescence upon depolarization and decreased fluorescence upon hyperpolarization (Epps et al., 1994, Chem Phys Lipids 69: 137-150). First passage myofibroblasts were plated on glass coverslips, allowed to attach, then incubated with 1 μM DiBAC₄(3) in control solution containing: NaCl (140 mM), KCl (5 mM), HEPES (10 mM), CaCl₂ (2.0 mM), MgCl₂ (1.4 mM) and glucose (10 mM) for 20 minutes. The cells were visualized at 400× on an inverted microscope (Nikon Canada). DiBAC_{4 (}3) was excited at 470 nm wavelength and emitted light at 525 nm was digitized and stored (Photon Technology International, Lawrenceville, N.J.). Fluorescence intensity was normalized to maximum fluorescence observed throughout the experiment. To ensure fluorescence intensity changes in response to altered membrane potential were occurring as expected, the cells were superfused with Tyrode's solution containing 1 μM DiBAC₄(3) and 1.5 mM KCl or 15 mM KCl. Under these control conditions, DiBAC₄(3) fluorescence decreased or increased indicating hyperpolarization or depolarization, respectively. For CT-1 experiments, CT-1 was diluted in control solution containing 1 μM DiBAC₄(3). After ensuring a stable fluorescence signal, the cells were superfused with the CT-1 containing solution for 4 minutes, followed by washout. Fluorescence was monitored for a total of 10 minutes.

Use of Pharmacologic Inhibitors

The use of pharmacologic inhibitors of signaling pathways has been widely practiced in determining signaling by a ligand. Tyrphostin B42 (AG490) was identified as an inhibitor of Jak2 in 1996 (Meydan et al., 1996, Nature 379: 645-648), and has been shown to have beneficial effects on various hematologic malignancies (Meydan et al., 1996; DeVos et al., 2000, Br J Haematol 109: 823-828). AG490 was shown to attenuate angiotensinogen mRNA production in response to CT-1 stimulation (Fukuzawa et al., 2000, Hypertension 35: 1191-1196). Genistein is a phytoestrogen that possesses tyrosine kinase inhibiting activity (Dixon and Genistein, 2002, Phytochemistry 60: 205-211). PD98059 is a highly specific inhibitor of MEK1/2, and therefore an inhibitor of p42/44 MAPK activation. It maintains its selectivity at concentrations as high as 50 μM (Davies et al., 2000, Biochem J 351: 95-105). SB203580 is a highly specific inhibitor of p38 MAPK with an IC₅₀ of 50 to 500 nM, depending on the isoform, and maintains its selectivity when used at 10 μM (Davies et al., 2000). Rapamycin is a highly specific inhibitor of mTOR and does not inhibit other kinases when used at 1 μM (Davies et al., 2000). LY294002 is a specific inhibitor of P13K, but also inhibits casein kinase 2 with equal efficacy. The IC₅₀ for LY294002 inhibition of P13K is 10 μM (Davies et al., 2000).

Results

Expression of CT-1 in Post-MI Heart.

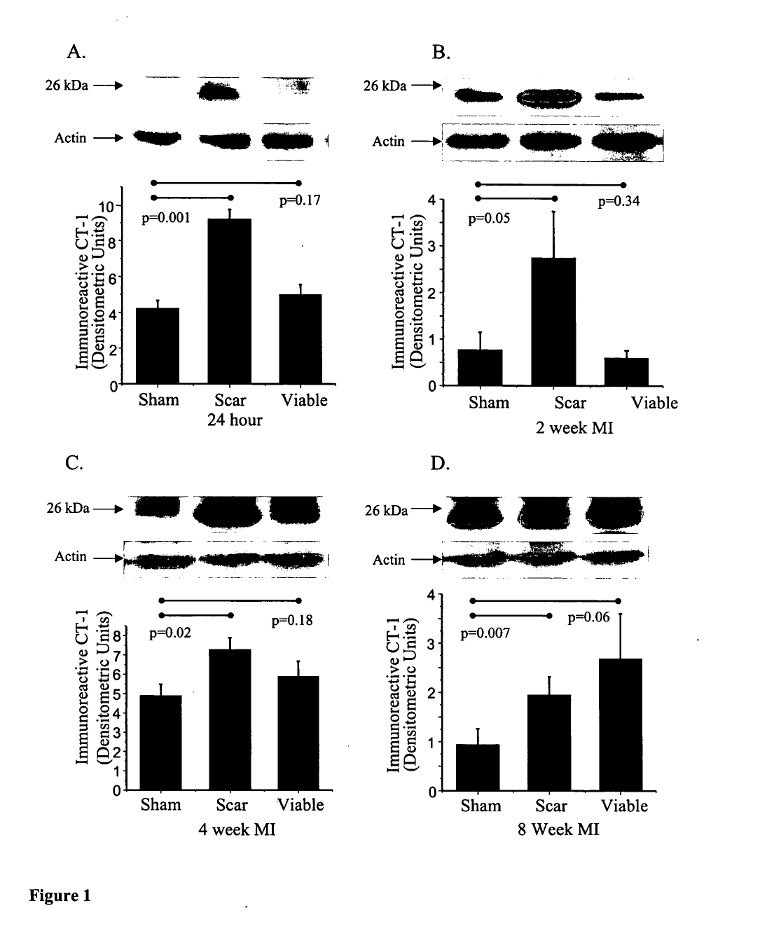

To examine the tissue expression of CT-1 in the setting of myocardial ischemic injury, we used the rat model of myocardial infarction. Ligation of the left coronary artery was performed in rats, and only those animals which developed an infarct occupying greater than 40% of the left ventricle were used for analysis. Tissue lysates from the infarct zone/scar, and remnant or viable left ventricle and the left ventricles of sham operated animals were prepared at 24 and 48 hours, and 2, 4 and 8 weeks after the ligation. These lysates were subjected to SDS-PAGE and Western blotting with specific CT-1 monoclonal antibodies. Elevated CT-1 expression (relative to sham and viable LV) was observed in tissue lysates from the infarct zone at the 24 hour time point, and elevated expression was observed up to 8 weeks after MI. At 8 weeks, elevated CT-1 was also observed in the viable LV, consistent with the development of left ventricular hypertrophy, as has previously been described (Dixon et al., 1990, Cic Res 66: 782-788; Makino et al., 1996, J Mol Cell Cardiol 28: 507-517) (FIG. 1). A graphic representation of relative CT-1 expression is shown in FIG. 2. Knowing that CT-1 was expressed in the infarct zone, we sought to determine the effects of CT-1 on isolated cardiac fibroblasts, the sole function of which is to maintain the extracellular matrix.

Phenotypic Modulation of Rat Cardiac Fibroblasts when Cultured.

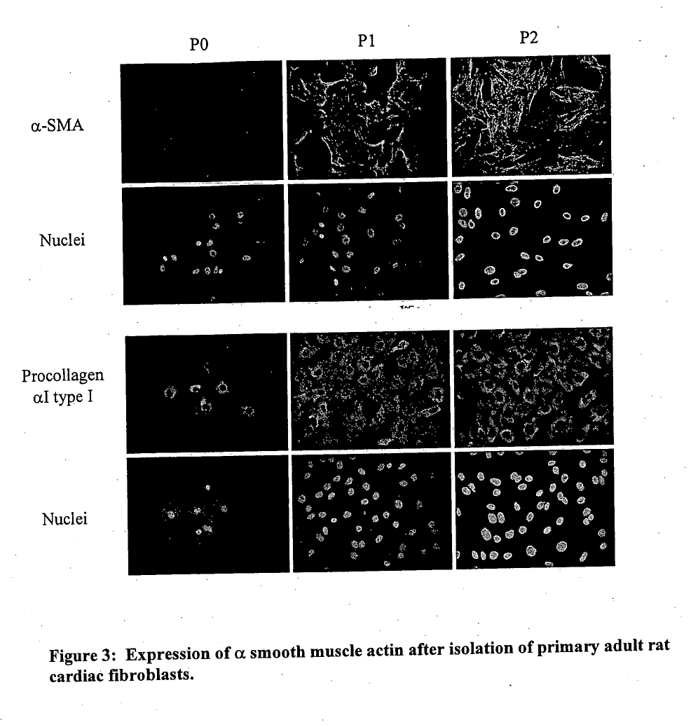

During myocardial wound healing, α-smooth muscle actin expressing myofibroblasts appear in the infarct zone. To determine if cultured cardiac fibroblasts undergo a phenotypic change to myofibroblasts when cultured, we isolated fibroblasts from the hearts of Sprague-Dawley rats and plated them on glass coverslips. After 1 day in culture, these cells are negative for α-smooth muscle actin, but stain positively for procollagen, indicating that they are fibroblasts. After the first passage (5-7 days after isolation), these cells stain positively for α-smooth muscle actin and procollagen, indicating that they are myofibroblastic cells (FIG. 3).

Signaling Pathways Activated by CT-1.

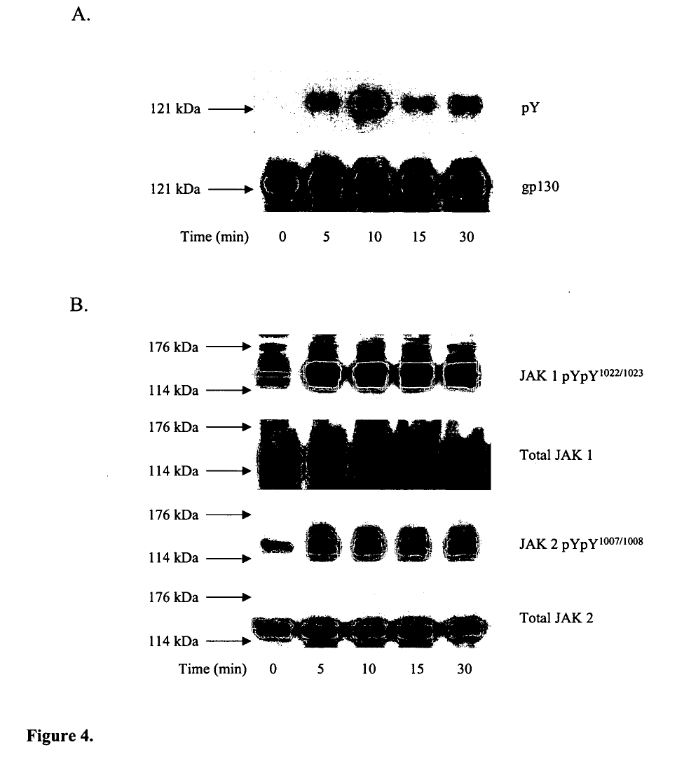

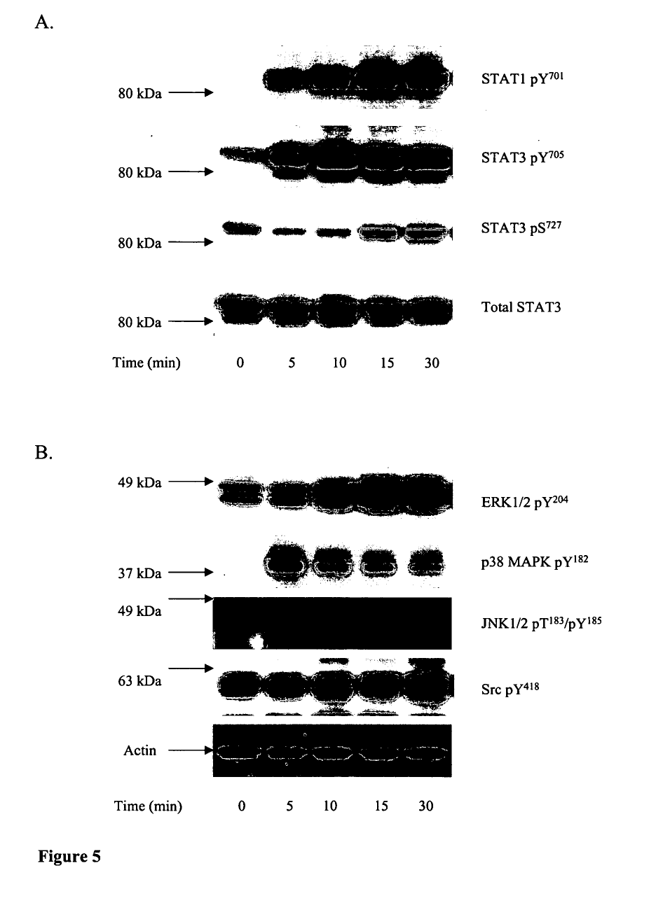

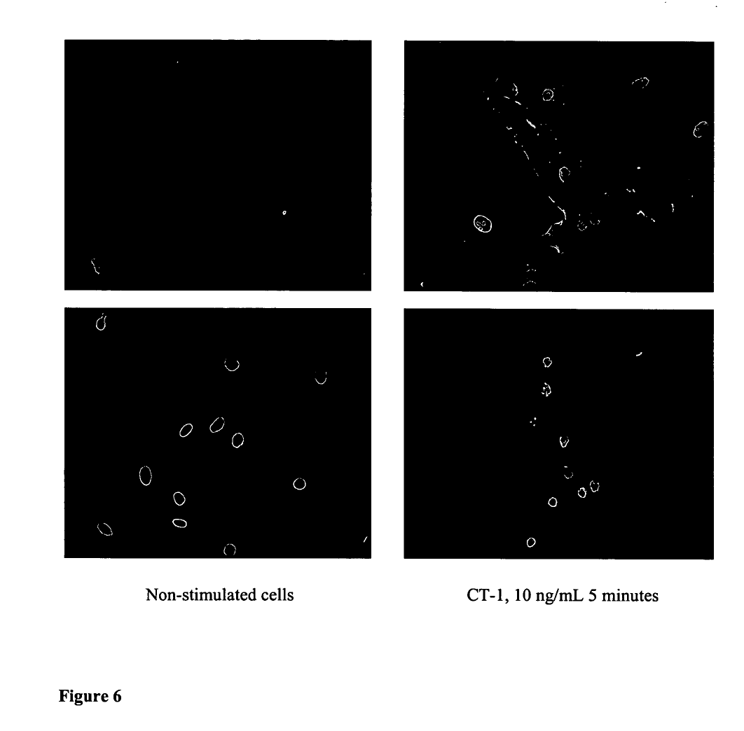

Cytokines of the IL-6 family typically activate the Jak pathway, which can subsequently activate multiple down-stream signaling pathways (Heinrich et al., 1998, Biochem J 334: 297-314; Rane and Reddy, 2000, Oncogene 20: 5662-5679). To screen for the signaling pathways activated by CT-1, cell lysates of cardiac fibroblasts stimulated with CT-1 were subjected to SDS-PAGE and Western blotting with phospho-specific antibodies to common intracellular signaling mediators. To examine phosphorylation of gp130, immunoprecipitation of gp130 from the cell lysates was first performed, followed by SDS-PAGE and Western blotting with anti-phosphotyrosine antibody. As expected, incubation of cardiac fibroblasts with CT-1 resulted in phosphorylation of gp130, Jak1, Jak2 (FIG. 4), STAT3, STAT1 and nuclear accumulation of STAT3 (FIG. 5a and FIG. 6). Phosphorylation of STAT5 or STAT6 was not observed (data not shown). Consistent with their enzymatic activation, we observed phosphorylation of p42/44 MAPK, p38 MAPK, JNK and Src (FIG. 5b).

Carditrophin-1 Induces Cardiac Fibroblast Proliferation.

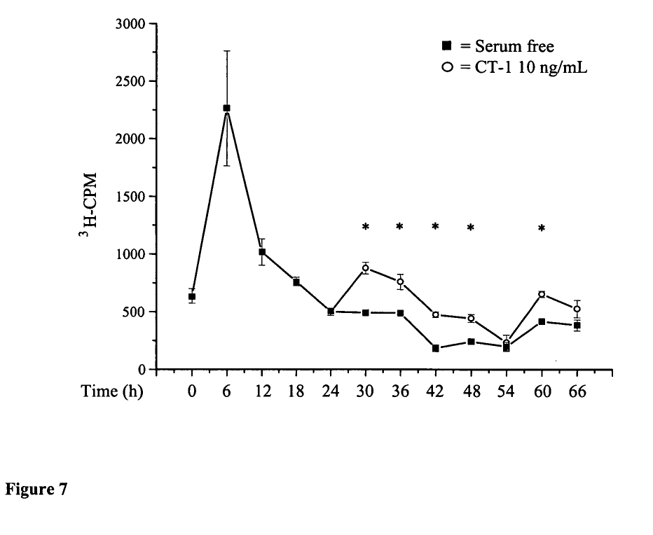

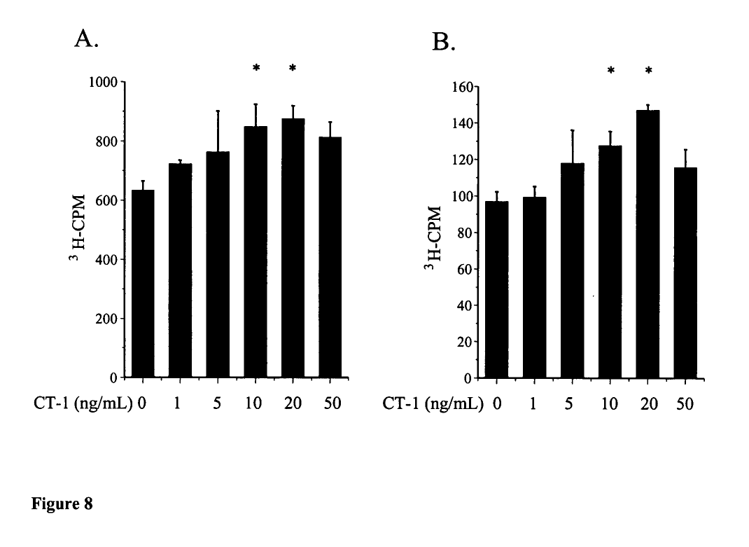

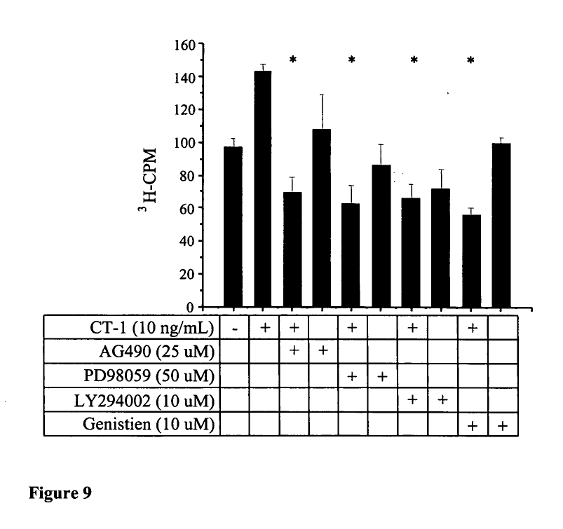

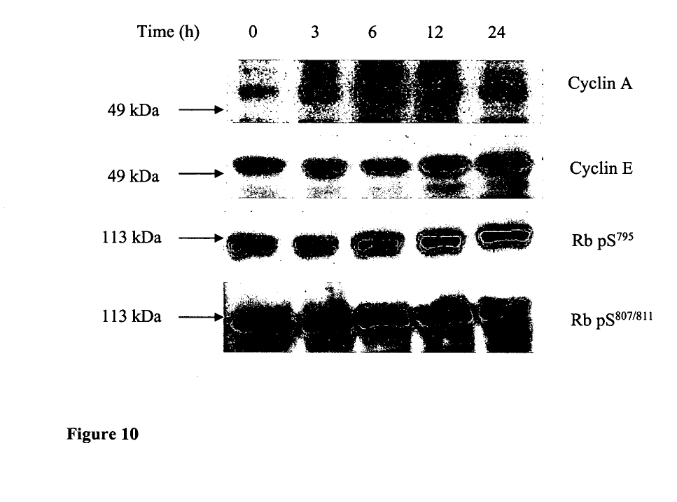

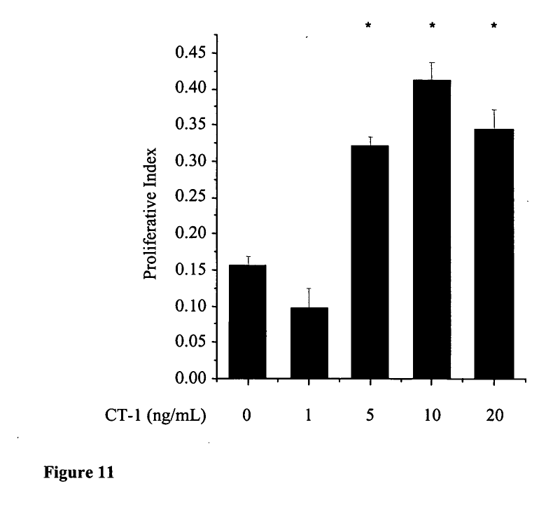

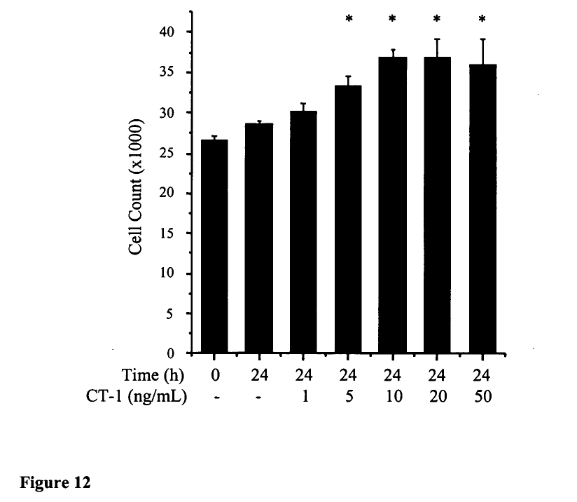

To determine if CT-1 possesses mitogenic properties for cardiac fibroblasts, we used incorporation of ³H-labeled thymidine as a measure of DNA synthesis. Cardiac fibroblasts were isolated from the ventricles of rats and incubated with CT-1 for specified times and at specified concentrations. FIG. 7 demonstrates that upon removal of serum, cardiac fibroblasts complete S-phase and become quiescent. Addition of CT-1 results in S-phase re-entry within 6 hours of stimulation (FIG. 7). Elevated incorporation of ³H thymidine was observed with as little as 1 ng/mL CT-1, and peak incorporation was observed at a dose of 10-20 ng/mL CT-1. (FIG. 8). CT-1 induced cell proliferation was dependent on Jak, P13-K, MAPK, and Src kinases (FIG. 9). To determine the effect of CT-1 on cell cycle regulatory proteins, we incubated cardiac fibroblasts with CT-1 and lysed them at specified times. CT-1 treatment resulted in an increase in expression of cyclins and hyperphosphorylation of the retinoblastoma protein (FIG. 10), which relieves its inhibition of E2F and allows activation of target genes and progression through the cell cycle (Stevens and Thangue, 2003, Arch Biochem Biophys 412: 157-169). CT-1 stimulation resulted in increased nuclear expression of PCNA, a DNA polymerase co-factor (FIG. 11). To determine if increased DNA synthesis was accompanied by an increase in cell number, we repeated the experiments in the absence of ³H-thymidine, detached the cells from the plates and counted the total number of cells. Increased incorporation of ³H-thymidine was accompanied by an increase in total cell number (FIG. 12). These results strongly support the hypothesis that CT-1 is a mitogen for primary adult rat cardiac fibroblasts.

Cardiotrophin-1 Induces Protein Synthesis in Cardiac Fibroblasts.

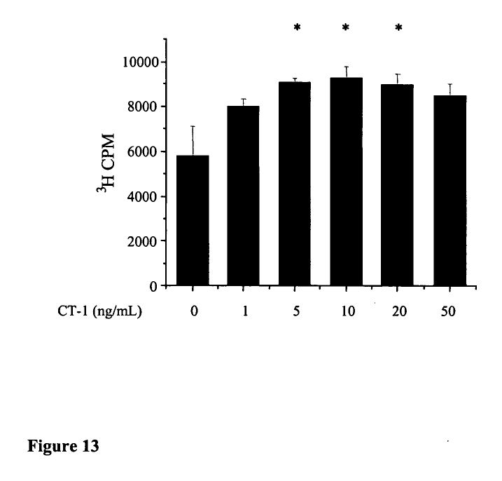

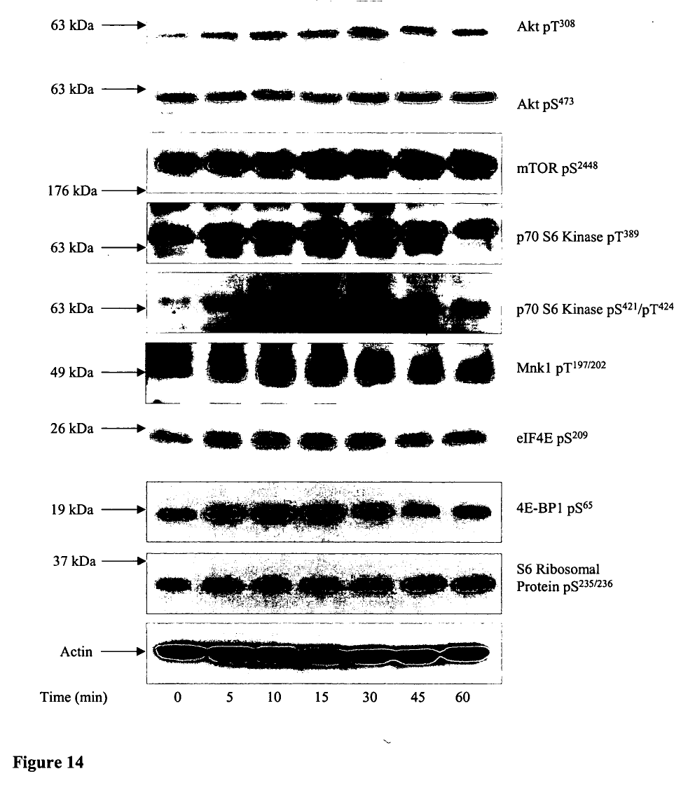

To determine the effect of CT-1 on protein synthesis in cardiac fibroblasts we measured incorporation of ³H-labeled leucine. Stimulation of cultured cardiac fibroblasts with CT-1 induced a dose-dependent increase in protein synthesis (FIG. 13). Since the activity of translational regulatory proteins is modified by phosphorylation, we used phospho-specific antibodies to determine if CT-1 directly influenced the activity of these regulatory proteins. Stimulation of cardiac fibroblasts with CT-1 induced an increase in phosphorylation of Akt at threonine 308 and serine 473, and increased phosphorylation of p70 S6 kinase at threonine 389 and serine 421/threonine 424, as well as phosphorylation of Mnk1, eIF4E, 4E-BP1 and S6 ribosomal protein (FIG. 14).

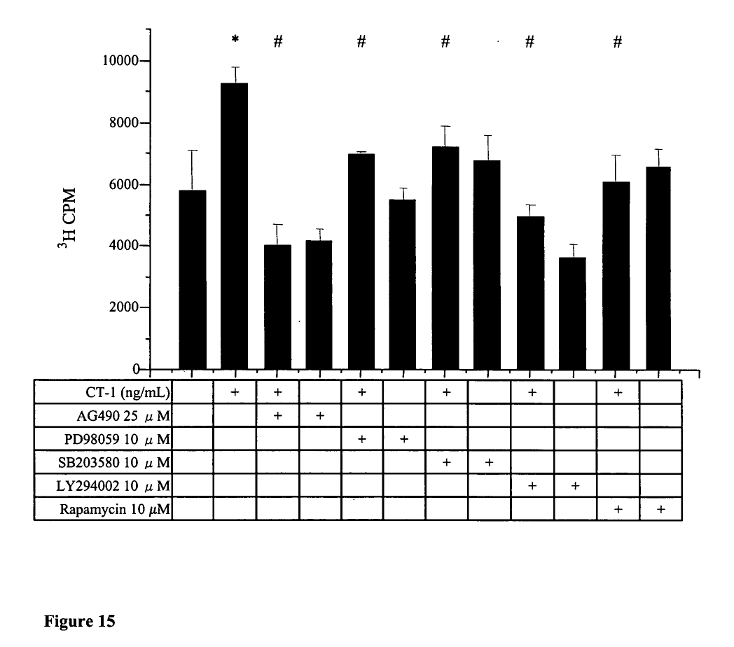

We then determined the effect of pharmacologic inhibitors on the activation of this signaling cascade. AG490, an inhibitor of Jak2 (Meydan et al., 1996, Nature 379: 645-648), suppressed CT-1 induced phosphorylation of Akt at both sites, p70 S6 kinase T³⁸⁹, Mnk1, eIF4E and 4E-BP1, but did not significantly affect CT-1 induced activation of ERK 1/2, p38 MAPK, mTOR, p70 S6 kinase S⁴²¹/T⁴²⁴, or S6 ribosomal protein. PD98059, an inhibitor of MEK 1/2 (Davies et al., 2000, Biochem J 351: 95-105) had the greatest inhibitory effect on basal and CT-1 induced phosphorylation of ERK 1/2, Mnk1, and eIF4E, but had a lesser effect on CT-1 induced phosphorylation of Akt, mTOR, p70 S6 kinase, 4EB-P1 and S6 ribosomal protein. SB203580, an inhibitor of p38 MAPK (Lee et al., 1999, Pharmacol Ther 82: 389-397), suppressed CT-1 induced phosphorylation of Akt, p70 S6 kinase and Mnk1, a finding that supports previous work demonstrating that p38 MAPK can activate Akt (Nomura et al., 2001, J Biol Chem 276: 25558-25567), p70 S6 kinase (Zhang et al., J Biol Chem 276: 20913-20923) and Mnk1 (Waskiewicz et al., 1997, EMBO J. 16: 1909-1920). As expected, LY294002, an inhibitor of P13K (Davies et al., 2000, Biochem J 351: 95-105), suppressed basal and CT-1 induced phosphorylation of Akt, mTOR, p70 S6 kinase, Mnk1, eIF4E, 4E-BP1 and S6 ribosomal protein. LY294002 also attenuated activation of ERK 1/2 and p38 MAPK suggesting that P13K participates in CT-1 induced activation of these signaling mediators. Rapamycin, an inhibitor of FRAP/mTOR (Davies et al., 2000), decreased basal and CT-1 induced phosphorylation of p70 S6 kinase and S6 ribosomal protein phosphorylation, and had a lesser effect on 4E-BPI phosphorylation (FIG. 15).

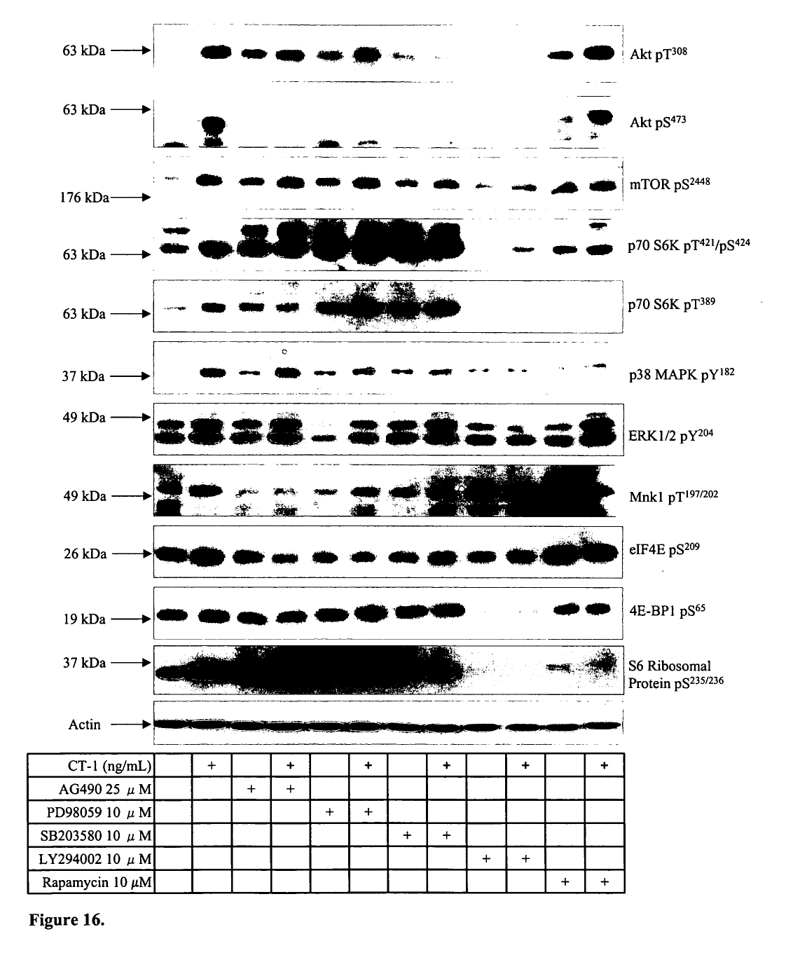

To determine if the inhibitory effect of these pharmacologic inhibitors impacted CT-1 induced protein synthesis, we again measured CT-1 induced ³H-leucine uptake in the presence of inhibitors of the Jak/STAT, P13K, p42/44 and p38 MAPK and mTOR pathways. CT-1 induced protein synthesis could be suppressed by co-incubation with AG490, PD98059, SB203580, LY294002 or Rapamycin (FIG. 16).

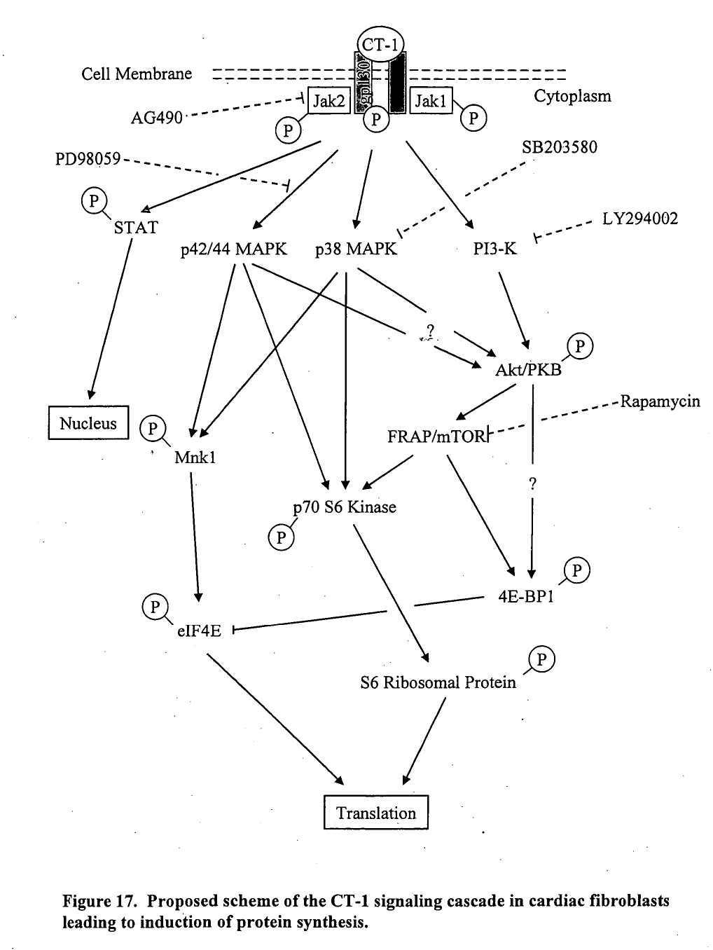

Our interpretation of how these pathways are integrated is shown in FIG. 17. These results demonstrate that CT-1 induced protein synthesis occurs through activation of typical translation regulatory proteins.

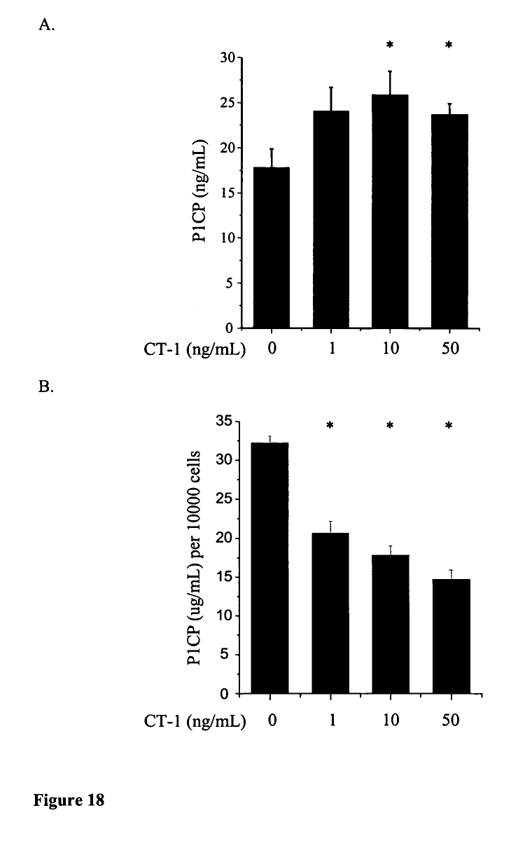

To examine synthesis of mature collagen, we utilized a radioimmunoassay to measure the expression of procollagen-1-carboxypropeptide (P1CP) in conditioned media from cardiac fibroblasts stimulated with CT-1. This method has been shown to correlate well with incorporation of ³H-proline into collagenase sensitive proteins (Nacher et al., 1999, Calcif Tissue Int 64: 224-228), but is much easier to perform. CT-1 stimulation of cardiac fibroblasts increased expression of P1CP in the culture media, indicating an overall increase in mature collagen synthesis. Since CT-1 also induces cardiac fibroblast proliferation, we normalized P1CP expression to the number of cells present at the end of the experiment. Although equal numbers of cells were loaded at the beginning of the experiment, the proliferative effect of CT-1 was much greater than its collagen synthetic effect resulting in an apparent decrease in overall mature collagen synthesis (FIG. 18). This has observation has important implications with reference to the effect of CT-1 on cardiac fibrosis in vivo, and suggests that CT-1 does not contribute to the overt cardiac fibrosis that follows MI, and may in fact dampen the effects other putative profibrotic agents.

Cardiotrophin-1 Induces Cardiac Fibroblast Migration.

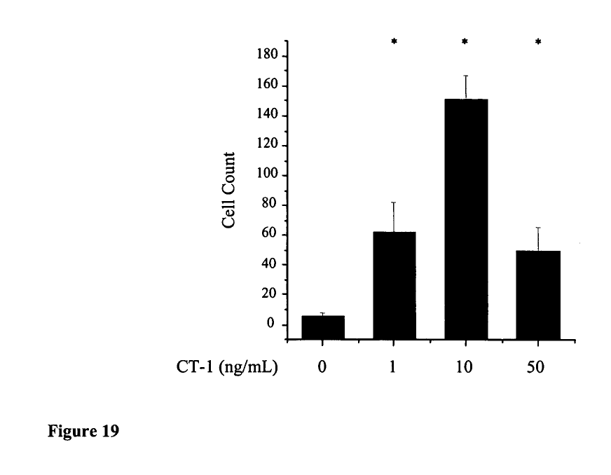

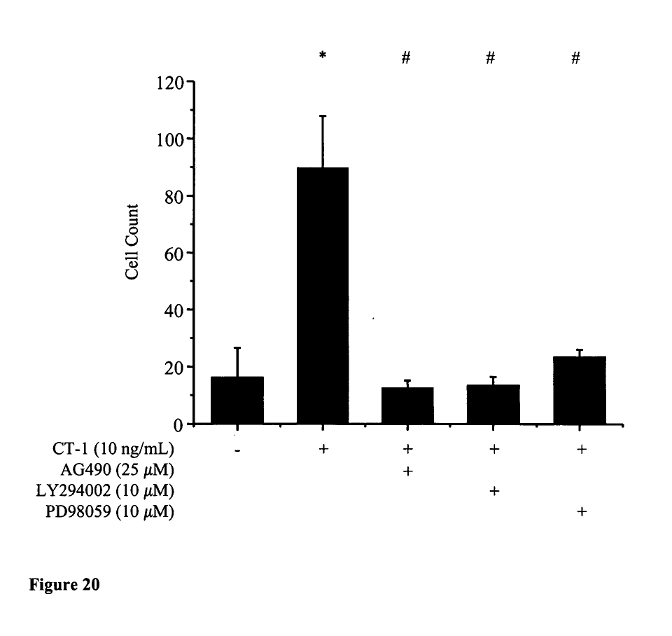

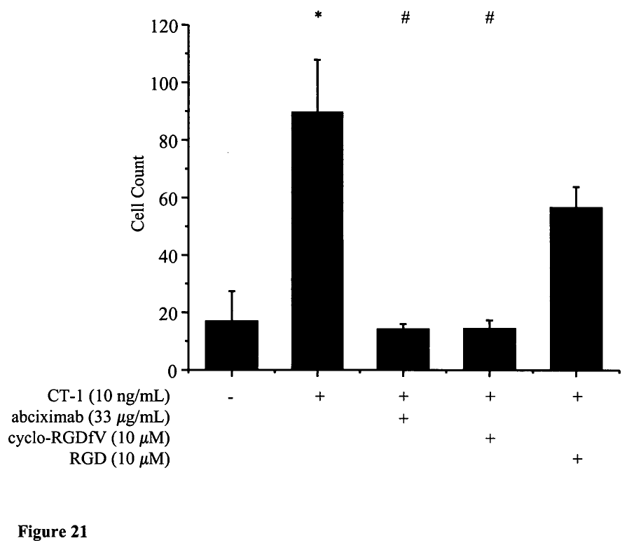

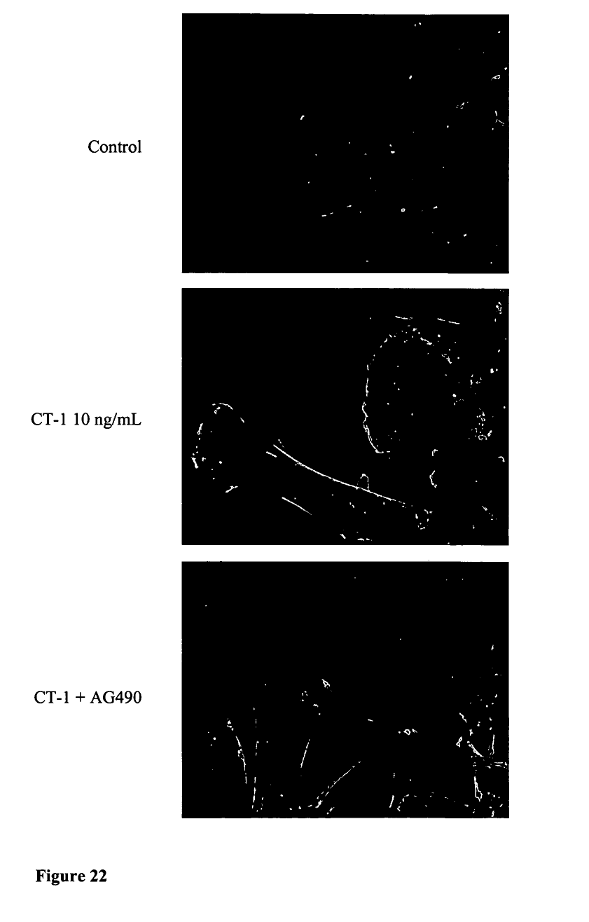

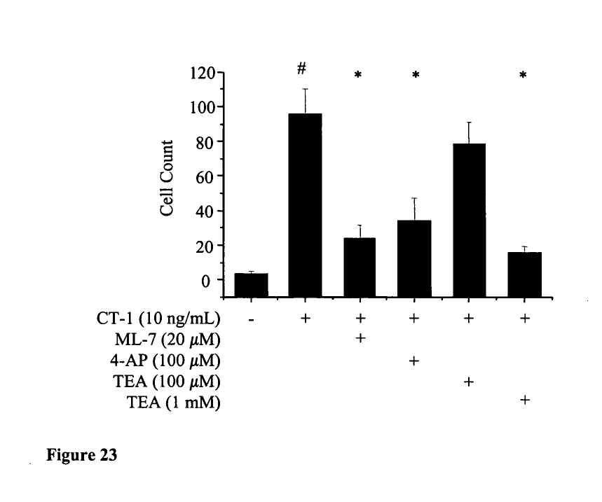

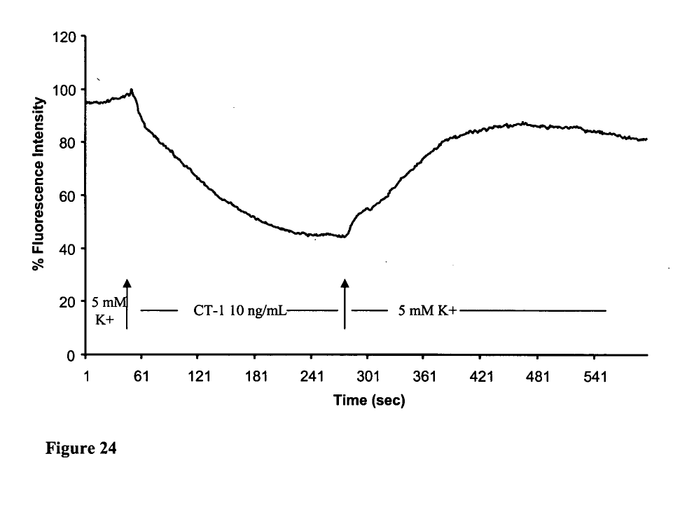

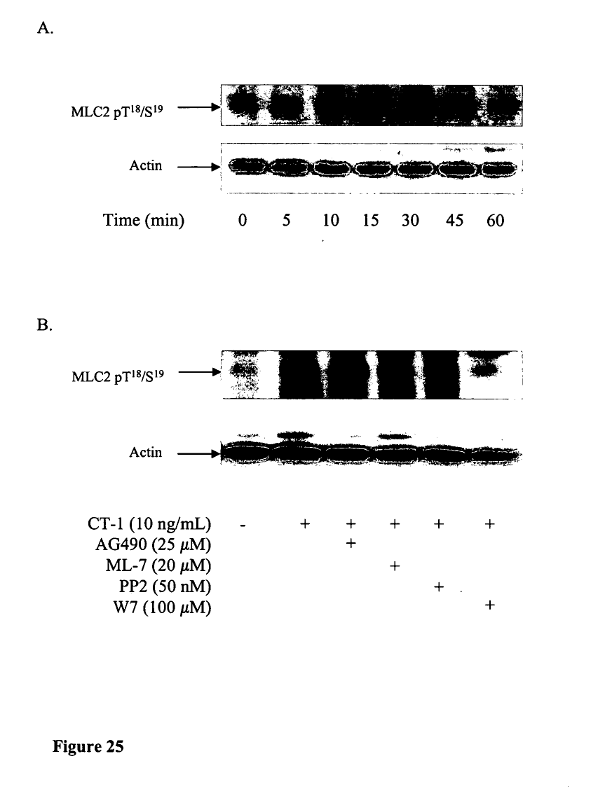

The infarct zone is repopulated by interstitial fibroblasts that infiltrate the scar after MI. To determine if CT-1 possesses chemoattractive properties, we used a Boyden chamber. Stimulation of cardiac fibroblasts with a CT-1 gradient induced cardiac fibroblast migration in a dose dependent manner (FIG. 19). Similar results were obtained when using the Costar Transwell® system (data not shown). To determine how the chemotactic response is transduced, we used specific inhibitors of intracellular signaling pathways. CT-1 chemotaxis is dependent on intracellular signaling pathways (FIG. 20) and integrin function (FIG. 21). Stimulation of migrating cardiac fibroblasts with CT-1 resulted in localization of the actin stabilizing protein, cortactin, at the leading edge of lamellipodia. This effect could be blunted by co-incubation with AG490 (FIG. 22). CT-1 induced migration is dependent on functioning potassium channels and myosin light chain kinase (FIG. 23). CT-1 induced a pronounced hyperpolarization of the cell, as indicated by decreased fluorescence of the voltage sensitive dye DiBAC₄(3) (FIG. 24). Since activation of myosin motors is required for effective cell migration, and CT-1 induced migration could be inhibited by co-incubation with a myosin light chain kinase inhibitor, we sought to determine the effect of CT-1 on the phosphorylation status of myosin light chain. Myosin light chain regulates the ATPase activity of myosin heavy chain, and thus overall contractile activity. As seen in FIG. 25a, CT-1 induced phosphorylation of myosin light chain at threonine 18/serine 19. Myosin light chain phosphorylation could be prevented by co-incubation with ML-7 or W7 but not AG490 or PP2 (FIG. 25b). This suggests that CT-1 induced phosphorylation of myosin light chain in cardiac myofibroblasts occurs through activation of calmodulin and not through activation of Src kinases. This also suggests that CT-1 induced cell membrane hyperpolarization is attended by an increase in intracellular calcium.

Discussion

Cardiotrophin-1 Expression in Post-MI Heart.

In this study we have examined the expression of CT-1 in the post-MI heart, and have observed elevated expression in the infarct zone at all time points examined. Additionally, we have observed elevated expression in the viable myocardium in the chronic phase of wound healing (8 weeks). Clearly, CT-1 is beneficial during the early period of post-MI wound healing, but in the long term may contribute to ventricular dilation and deterioration of ventricular function. It is known that CT-1 is cardioprotective from ischemia-reperfusion injury, even when added at the point of reoxygenation (Brar et al., 2001, Cytokine 16: 93-96; Brar et al., 2001, Cardiovasc Res 51: 265-274; Liao et al., 2002, Cardiovasc Res 53: 902-910). Therefore early expression of CT-1 in the ischemic myocardium may represent an adaptive, protective phenomenon that is beneficial in reducing myocyte loss and inducing hypertrophy of remaining myocytes so that overall ventricular function is maintained. However, it is suggested that CT-1 expression in the late phase of wound healing and the onset of heart failure may contribute to ventricular dilation by inducing hypertrophy of myocytes where sarcomeres are arranged in series (eccentric hypertrophy), rather than in parallel as is seen in concentric cardiac hypertrophy (Wollert et al., 1996, J Biol Chem 19: 9535-9545), although this hypothesis remains to be proven.

In addition to possibly protecting myocytes, the observation that CT-1 induces migration and proliferation of myofibroblasts suggests that localized expression in the infarct zone may act to stimulate migration of cardiac fibroblasts and other cells from the surrounding viable myocardium into the infarct zone, thereby initiating repopulation of the scar with cells. It is known that bone marrow mesenchymal stem cells migrate to the scar after myocardial infarction (Orlic et al., 2003, Pediatr Transplant 7 Suppl 3: 86-88). We speculate that the expression of CT-1 in the infarct zone may provide a critical migratory stimulus for these cells, and may even induce (with other mediators) their differentiation to a myofibroblastic phenotype. The bulk of evidence gathered in the past 5 years which is aimed at muscle cell regrowth in the healed infarct scar indicates that a cellular scar confers improved ventricular performance compared a “hypocellular” or native infarct scar, irregardless of the cell type placed in the scar (Reffelmann and Kloner, 2003, Cardiovasc Res 58: 358-368; Li et al., 1999, J Mol Cell Cardiol 31: 513-522; Li et al., 1996, Ann Thorac Surg 62: 654-660; Taylor et al., 1998, Nat Med 4: 929-933). Infarct scar specific CT-1 expression may act to induce a more cellular phenotype through induction of cell proliferation and may antagonize the effects of angiotensin and TGF-β, both of which are expressed in the scar and act to induce cell quiescence (Bouzegrhane and Thibault, 2002, Cardiovasc Res 53: 304-312; Petrov et al., 2000, J Renin Angiotensin Aldosterone Syst 1: 342-352; Sigel et al., 1994, Mol Cell Biochem 141: 145-151) and unpublished observations]. Indeed, a common theme in explanation of the beneficial effects of popular ACE inhibiting agents (angiotensin-suppressing drugs) is that while angiotensin is suppressed, other systems are derepressed i.e., ACE inhibition prevents the destruction of bradykinin (Liu et al., 1997, J Clin Invest 99: 1926-1935). Thus the upregulation of a parallel system is invoked and yields a net benefit to the post-MI heart. We suggest that this mechanism may be an additional beneficial effect of angiotensin antagonism insofar as the CT-1 stimulus is allowed to act unopposed. In addition to opposing the action of these mediators, CT-1 expression may antagonize TNF-α induced myocyte apoptosis (Benigni et al., 1996, Am J Pathol 149: 1847-1850; Mann, 2002, J Card Fail 8: S379-S386), thereby preserving overall ventricular performance.

Although the heart is the major source of circulating CT-1 in humans (Asai et al., 2000, Biochem Biophys Res Commun 279: 320-323), the cellular source of CT-1 in the post-MI heart is not clear. While induction of CT-1 mRNA has been shown in cardiac fibroblasts (Sano et al., 2000, J Biol Chem 275: 29717-29723) and myocytes (Pan et al., 1999, Circ Res 84: 1127-1136; Funamoto et al., 2000, J Mol Cell Cardiol 32: 1275-1284; Hishinuma et al., 1999, Biochem Biophys Res Commun 264: 436-440) in vitro in response to a variety of stimuli, it is not clear which cell type is primarily responsible for in vivo myocardial CT-1 expression. It is possible that inflammatory cells contribute to CT-1 expression, particularly early on in the course of post-MI wound healing. Irrespective of the cellular source, activation of the gp130 signaling cascade is required for an appropriate myocardial response to injury that allows survival of the animal (Hirota et al., 1999, Cell 97: 189-198). It was suggested that cardiac myocyte hypertrophy produced by angiotensin is mediated through CT-1 (Sano et al., 2000, Biochem Biophys Res Commun 269: 798-802) however recent studies in humans do not support an upstream connection to angiotensin (Talwar et al., 2002, Clin Sci (Lond) 102: 9-14). Conversely norepinephrine, which is locally and systemically activated after MI (Schoming, 1990, Circulation 82: II 13-II 22), is known to elevate CT-1 expression in cardiac myocytes in vitro and in vivo via a cAMP response element in the 5′ flanking region of the CT-1 gene (Funamoto et al., 2000, J Mol Cell Cardiol 32: 1275-1284). This finding suggests the involvement of α-adrenergic stimulation in post-MI CT-1 expression.

The infarct scar has been shown to be a vascular structure and myocardial wound healing after infarction invokes a significant upregulation of angiogenesis in the infarct zone (Sun and Weber, 2000, Cardiovasc Res 46: 250-256). It is suggested that CT-1 induced STAT3 activation as observed in the post-MI heart may act to initiate angiogenesis as STAT3 activation is known to induce VEGF-dependent endothelial tubule formation and vasculogenesis in vivo (Osugi et al., 2002, J Biol Chem 277: 6676-6681).

In this study we have examined the effects of CT-1 on cardiac myofibroblasts to investigate the possible involvement of this IL-6 family member cytokine in remodelling post-MI heart. We are able to confirm that CT-1 activates JAK 1 and 2 (but not JAK3 or Tyk2), leading to the phosphorylation of STAT3 and STAT1 which then translocate to the nucleus of myofibroblasts. Others have shown that upon gp130 activation, rapid negative regulation of JAKs occurs via induction of suppressor of cytokine signaling 3 (SOCS3) in heart (Yasukawa et al., 2001, J Clin Invest 108: 1459-1467), and thus a balance between positive and negative regulatory loops is attained. While CT-1 exerts cardioprotective effects (Wollert et al., 1996, J Biol Chem 271: 9535-9545; Hirota et al., 1999, Cell 97: 189-198), myocardial expression of CT-1 is increased in heart failure models (Aoyama et al., 2000, J Mol Cell Cardiol 32: 1821-1830; Jougasaki et al., 2000, Circulation 101: 14-17), and in plasma (Talwar et al., 1999, Biochem Biophys Res Commun 261: 567-571) and cardiac tissues of patients with heart failure (Zolk et al., 2002, Circulation 106: 1442-1446). It is becoming clear that CT-1 expression generally precedes the development of pathological hypertrophy (Ishikawa et al., 1999, J Hypertens 17: 807-816). As mentioned previously, this may in fact be a beneficial event and required for an appropriate myocardial response to injury by activating the gp130 signaling cascade and inhibiting myocyte apoptosis (Hirota et al., 1999, Cell 97: 189-198).

Significance of the Mitogenic Effect of Cardiotrophin-1.

Accumulating evidence suggests that a cellular scar is better than a hypocellular scar. The “cellular cardiomyoplasty” literature has shown that irregardless of the cell type that is placed in the scar, ventricular performance is improved, even though these cells may not contribute to synchronous ventricular contraction (Reffelmann and Kloner, 2003, Cardiovasc Res 58: 358-368). Expression of CT-1 in the infarct zone is likely beneficial given the potent induction of cell proliferation and migration. In so doing, CT-1 acts to induce proliferation of and migration of fibroblasts from adjacent viable myocardium thereby repopulating and maintaining the cellularity of the scar. Additionally, CT-1 may act to maintain a proliferative, migratory phenotype and oppose the actions of angiotensin and TGF-β which act to induce a quiescent, contractile, hypersynthetic myofibroblast phenotype (Serini and Gabbiani, 1999, Exp Cell Res 250: 273-283; Dugina et al., 2001, J Cell Sci 114: 3285-3296; Bouzegrhane and Thibault, 2002, Cardiovasc Res 53: 304-312). LIF, another member of the IL-6 family of cytokines which induces a similar signaling pattern as CT-1, has been shown to reduce collagen expression and antagonized the switch to a myofibroblast phenotype (Wang et al., 2002, J Mol Cell Cardiol 34: 1309-1316). Although we did not specifically address the effect of CT-1 on cardiac fibroblast phenotype, it is likely that CT-1 has a similar effect, which again supports the notion that CT-1 has beneficial effects on cardiac fibroblast function in the post-MI heart.

The transcription factor STAT3 has been shown to induce VEGF dependent myotube formation when overexpressed (Osugi et al., 2002J Biol Chem 277: 6676-6681). Although this may represent another effect of CT-1 signaling in post MI heart, namely angiogensis, to date there is no information regarding the effect of CT-1 on endothelial cell or smooth muscle cell proliferation.

Role of Cardiotrophin-1 in Cardiac Fibroblast Protein Synthesis.

Our results show that CT-1 induces protein synthesis in cardiac fibroblasts through typical signaling pathways. Our interpretation of how these pathways may be integrated is shown in FIG. 15. Proteins that could be induced by CT-1 stimulation include extracellular matrix proteins (collagens, fibronectin, tenascin etc), proteins involved in regulation of cell cycle (cyclins, proliferating nuclear antigen, etc) and proteins required for cell adhesion and migration (primarily integrins) (Nurse, 2002, Chembiochem 3: 596-603; Danen and Yamada, 2001, J Cell Physiol 189: 1-13; Willems et al., 1996, J Pathol 179: 321-325). Our results have shown that the collagen synthetic stimulus of CT-1 is modest without cell number normalization and actually is associated with a reduction of collagen secretion if the normalized data are considered (FIG. 18). These findings indicate that CT-1 may not possess a significant profibrotic stimulus insofar as there is not a significant induction of fibrillar collagen synthesis. While this finding appears to be contradictory to other published data (Tsuruda et al., 2002, Circ Res 90: 128-134) the differences may be due to one of the following extenuating experimental parameters: i) the current dataset is presented in both raw and normalized forms, and it is the former subset that agrees with other published data, which also is not normalized to cell number; ii) we measured collagen synthesis by procollagen-1-carboxy propeptide (P1CP) expression in culture media, as opposed to simple incorporation of ³H-proline; iii) the current data were sourced from samples of culture medium as opposed to cell lysates. It is well known that collagen synthesis is a discoordinate process in cardiac myofibroblasts and therefore changes in collagen type I mRNA abundance may not accurately reflect changes in protein secretion (Besse et al., 1994, Am J Physiol 267: H2237-H2244), and most importantly, collagen secretion is mediated through a complex series of steps involving extensive post-secondary modification of procollagen monomers, self-winding of the triple helices, and finally cleavage of both N- and C-terminal polypeptides. In cases whereby myofibroblasts are unstimulated or there is a deficiency of cofactors, a high proportion of collagen monomers are cycled within the cell, degraded shortly after synthesis and therefore never reach the point of secretion (Eleftheriades et al., 1995, J Mol Cell Cardiol 27: 1459-1473). Thus the determination of secreted collagen is the preferred method for ascertaining net collagen synthesis in cardiac myofibroblasts, and we feel that the advantages conferred by use of the P1CP method outweigh those that may be realized by addressing collagen type I mRNA expression or incorporation of ³H-proline alone. On balance, we suggest that the proteins that are generated in response to the CT-1 stimulus are involved in induction of cell functions (such as proliferation or migration) other than synthesis of secreted ECM proteins.

Significance of Cardiotrophin-1 Induced Cardiac Fibroblast Cell Migration.

The observation that CT-1 induces cardiac fibroblast migration coupled with infarct scar specific expression of CT-1 suggests that it contributes to repopulation of the scar as mentioned previously. Although CT-1 does appear to have a chemotatic effect on porcine coronary artery vascular smooth muscle cells (data not shown), there is conflicting evidence for the role of IL-6 family cytokines on angiogensis. Whereas LIF may act to reduce angiogenesis in vitro (Pepper et al., 2003, J Cell Sci 108: 73-83), OSM induces angiogenesis in vivo and in vitro and is chemoattractant for human microvascular endothelial cells (Vasse et al., 1999, Arterioscler Thromb Vasc Biol 19: 1835-1842).

Induction of cell migration by CT-1 requires cytoskeletal reorganization, increased focal adhesion dynamics and increased contractile activity. We have shown that CT-1 induces myosin light chain phosphorylation, which leads to increased actin-mysoin cross-bridge cycling and contractile activity. The observation that migration was dependent on intact potassium channel function, coupled with the observation that CT-1 induces myofibroblast cell membrane hyperpolarization, indicates that the mechanism of CT-1 induced migration is similar to that observed with other non-excitable cells (Wang et al., 2000, Am J Physiol Cell Physiol 278: C303-C314; Negulescu et al., 1994, Proc Natl Acad Sci USA 91: 2873-2877; Nelson and Quayle; Rao et al., 2001, Am J Physiol 282: C885-C898). Hyperpolarization of the cell membrane would increase the driving force for calcium entry through non-selective cation channels. Although our studies have not specifically addressed calcium flux, there is functional evidence that these cells express these channels (Kamkin et al., 2003, Cariovasc Res 57: 793-803).

CT-1 induced hyperpolarization of the cell membrane, through activation of a potassium channel, is integral to cell functions such as migration, as indicated by our data. Although we haven't specifically demonstrated an increase in intracellular calcium content, it is implied through the observation of increased calmodulin-dependent myosin light chain phosphorylation (FIG. 25). Cell membrane hyperpolarization can increase intracellular calcium through an increase in the driving force for calcium flux through capacitive cation channels. This increased intracellular calcium content acts to activate contractile machinery through activation of myosin light chain kinase. This effect is required for cell function such as migration and contraction and for vesicular transport and secretion of proteins from the cell (Parekh and Penner, 1997, Physiol Rev 77: 901-930).

### Conclusions

In conclusion, we have shown that cardiotrophin-1 is expressed in the infarct zone after permanent coronary artery ligation, and that it induces migration, proliferation and protein synthesis in cardiac fibroblasts. Taken together, these findings indicate that CT-1 plays a role and is beneficial in post-MI wound healing. To confirm this, gain of function and loss of function experiments need to be done. We plan to do an experiment involving administration of recombinant CT-1 to rats after coronary artery ligation, looking for changes in mortality, infarct size, ventricular performance, cellularity and collagen deposition. A CT-1 knock out mouse exists (Oppenheim et al., 2001, J Neurosci 21: 1283-1291) which would be an ideal tool for studying loss of function experiments again looking at the same endpoints. The knock-out model could also be used to determine the role of CT-1 in the accumulation of mesenchymal stem cells in the infarct zone. The hyperpolarizing effect of CT-1 on cell membrane is an intriguing observation that requires further investigation. As myofibroblasts are not only hypersynthetic, but also contractile cells that mediate contraction of the scar (scar thinning), the effect of CT-1 on cell shortening needs to be examined and compared with known mediators of myofibroblast function. It is likely that CT-1 acts to inhibit cell contraction (which is likely stimulated by mediators such as angiotensin and TGF-β).

According to another aspect, there is provided a method of identifying mimetics of CT-1, that is, of identifying compounds capable of mimicking the biological effects of CT-1. As will be appreciated by one of skill in the art, this involves screening for example compounds, biomolecules and small molecules for the ability to mimic the biological effects of CT-1, for example, activating the gp130 cascade or promoting BMSC migration. In these embodiments, candidate mimetic compounds are assayed for the ability to mimic the biological effects of CT-1 as discussed above using methods described herein. The methods may be adapted for suitability in combination with high throughput assays where appropriate. In another embodiment of the invention, there are provided CT-1 mimetics isolated by the above-described method.

In some embodiments, CT-1, a bioactive fragment thereof or a mimetic thereof the may be combined with other compounds or compositions known in the art such that the is a pharmaceutical composition in the form of, for example, a pill, tablet, liquid, film or coating using means known in the art and as discussed below.

It is of note that the discussed above may be prepared to be administered in a variety of ways, for example, by osmotic mini-pump, IP or local injections, or possibly by viral vectors that are built to yield tissue specific binding (i.e., incorporation a promoter with gene-specific elements to the target tissue such as b-MHC promoter in heart muscle).

It is of note that as discussed herein, the above-described pharmaceutical composition may be arranged to be delivered at an effective dosage. As will be appreciated by one of skill in the art, the total dosage will vary according to the weight, health and circumstances of the individual.

In some embodiments, the above-described pharmaceutical composition at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In other embodiments, the above-described pharmaceutical composition at concentrations or dosages described above may be encapsulated for delivery. Specifically, the pharmaceutical composition may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery of the. Alternatively, the delivery vehicle may be suspended in saline and used as a nanospray for aerosol dispersion onto an area of interest. Furthermore, the delivery vehicle may be dispersed in a gel or paste, thereby forming a nanopaste for coating a tissue or tissue portion or impregnated on a gauze or other similar material.

In yet other embodiments, CT-1, a bioactive fragment thereof or a mimetic thereof is used in combination with another known compound, for example, a targeting molecule. In yet other embodiments, CT-1, a bioactive fragment thereof or a mimetic thereof may be used in combination with.

In other embodiments, a pharmaceutical composition comprising an effective amount CT-1, a bioactive fragment thereof or a mimetic thereof as discussed above is administered to a patient in need thereof. The patient may be an animal, for example, a mammal, for example a human. Specifically, a therapeutic amount of the pharmaceutical composition is applied to the site of a wound, for example, but by no means limited to, on the skin, heart, lung, kidney, liver, teeth and gums or skeletal muscle of the patient. Application of CT-1, a bioactive fragment thereof or a mimetic thereof will accomplish at least one of the following: promote bone marrow stem cell migration to the wound site, reduce collagen deposition at the wound site, prevent excessive growth of remnant myocytes and reduce fibrosis.

While the preferred embodiments of the invention have been described above, it will be recognized and understood that various modifications may be made therein, and the appended claims are intended to cover all such modifications which may fall within the spirit and scope of the invention.

1. A method of promoting wound healing comprising administering to a patient in need thereof an effective amount of CT-1, a bioactive fragment of CT-1 or a mimetic thereof.

2. The method according to claim 1 wherein an effective amount of CT-1 is administered.

3. The method according to claim 1 wherein the wound is a cardiac infarct scar.

4. A pharmaceutical composition comprising an effective amount of CT-1, a bioactive fragment of CT-1 or a mimetic thereof.

5. Use of an effective amount of CT-1, a bioactive fragment of CT-1 or a mimetic thereof for promoting wound healing.
